(19) United States
(12) Patent Application Publication
(10) Pub. No.: US 2006/0228431 A1
(43) Pub. Date: Oct. 12, 2006

(54) Nutriceutical tea

(76) Inventors: David J. Eben, Stamford, CT (US); Bradley S. Miller, Lawrenceville, NJ (US)

Correspondence Address: AMSTER, ROTHSTEIN & EBENSTEIN LLP, 90 PARK AVENUE, NEW YORK, NY, 10016, US

(21) Appl. No.: 11/101,137

(22) Filed: Apr. 7, 2005

(51) Int. Cl. A61K 36/82; A61K 36/752; A61K 36/534; A61K 36/28; A61K 36/73; A61K 31/737; A61K 31/7008

(52) U.S. Cl. 424/729; 424/747; 424/764; 514/54; 514/62; 424/765; 424/736; 424/778; 424/641; 424/638; 424/702; 424/646; 514/52; 514/251; 514/276; 514/167; 514/350; 514/356; 514/393; 514/474

(57) ABSTRACT

Provided are compositions of plant material and a purified nutritional supplement or pharmaceutical adhering to the plant material with a binder. Also provided are potable beverages resulting from steeping or brewing these compositions. Further provided are delivery systems for a nutritional supplement or pharmaceutical having the above composition in a tea bag, where the nutritional supplement or pharmaceutical is in a quantity suitable for unit dose administration. Additionally provided are methods of ingesting a nutritional supplement or pharmaceutical by drinking the beverage resulting from steeping or brewing the above composition.

### BACKGROUND OF THE INVENTION

(1) Field of the Invention

The present invention generally relates to tea. More specifically, the invention relates to compositions and methods for ingesting nutritional supplements or pharmaceuticals incorporated into a tea.

(2) Description of the Related Art

Several tea preparations that provide a nutritional component are known. See, e.g., U.S. Pat. Nos. 6,277,428; 6,660,308; and 6,210,738; and U.S. Patent Publications 2004/0112395; 2003/0069202; 2004/0071855; and 2004/0053884. However, none provides a nutritional supplement or pharmaceutical adhering to the plant material, which can be used as a loose tea or in a tea bag. Such a composition is desirable because it provides for a convenient, familiar way of ingesting the nutritional supplement or pharmaceutical. The present invention satisfies that need.

### SUMMARY OF THE INVENTION

Accordingly, the inventors have discovered that nutritional supplements or pharmaceuticals can be usefully incorporated into teas when combined with a suitable binder.

Thus, in some embodiments, the invention is directed to compositions suitable for steeping or brewing to form a potable beverage. The compositions comprise plant material and a purified nutritional supplement or pharmaceutical adhering to the plant material with a binder.

In other embodiments, the invention is directed to potable beverages resulting from steeping or brewing the above composition.

The invention is additionally directed to delivery systems for a nutritional supplement or pharmaceutical. The delivery systems comprise the above composition in a tea bag, where the nutritional supplement or pharmaceutical is in a quantity suitable for unit dose administration.

Additionally, the invention is directed to methods of ingesting a nutritional supplement or pharmaceutical. The methods comprise drinking the beverage resulting from steeping or brewing the above composition.

### DETAILED DESCRIPTION OF THE INVENTION

The present invention is based in part on the inventor's discovery that purified nutritional supplements and pharmaceuticals can be formulated into a tea by adhering the supplement or pharmaceutical to the plant material in the tea with a binder.

Thus, in some embodiments, the invention is directed to compositions suitable for steeping or brewing to form a potable beverage. The compositions comprise plant material and at least one purified nutritional supplement or pharmaceutical adhering to the plant material with a binder.

As used herein, unless otherwise indicated, the term “tea” means any plant material (including fungus, e.g., mushrooms) that can be steeped or brewed to form a potable beverage. When followed by “(Camillia sinensis)”, “tea” means comprising leaves from that species. “Green tea”, “black tea”, and “white tea” are Camillia sinensis leaves prepared as is known for those tea types.

As used herein, the term “purified” means substantially free of contaminants, other than added pharmaceutical excipients (e.g., binders, inert diluents, edible carriers, etc.) Preferably, the purified nutritional supplement or pharmaceutical component of the purified composition is at least 50% of the composition (exclusive of excipients); more preferably, the purified nutritional supplement or pharmaceutical component is at least 75%; even more preferably at least 90%; and most preferably at least 95% of the composition (exclusive of excipients).

The present compositions can utilize any plant material that can be steeped or brewed to form a potable beverage. Nonlimiting preferred examples include tea (Camillia sinensis) leaves, jasmine flowers, chamomile flowers, peppermint leaves, spearmint leaves, ginger, rose hips, orange peels, lemon grass, Roobois, Echinacea, and ginseng. In some preferred embodiments, the plant material is tea (Camillia sinensis) leaves, for example green tea, black tea, or white tea. Such Camillia sinensis preparations can be decaffeinated, by methods known in the art.

In some preferred embodiments of these compositions, the at least one nutritional supplement or pharmaceutical is a nutritional supplement. As used herein, a nutritional supplement is a naturally occurring chemical compound useful for normal functioning of a human or animal. Nonlimiting examples of nutritional supplements are glucosamine, chondroitin, zinc, vitamin C, vitamin A, vitamin D, vitamin E, vitamin K, thiamine, riboflavin, niacin, vitamin B6, folic acid, vitamin B12, biotin, pantothenic acid, calcium, iron, magnesium, selenium, copper, manganese, chromium, potassium, methyl sulfonyl methane, P57, and lycopene. In some preferred embodiments, the nutritional supplement is glucosamine or chondroitin.

There can comprise more than one purified nutritional supplement or pharmaceutical in these compositions. Potentially, two, three, four, or more nutritional supplements or pharmaceuticals can be present. In some embodiments, the purified nutritional supplement or pharmaceutical is part of a multivitamin composition adhering to the plant material with a binder. In other preferred embodiments, the composition comprises both glucosamine and chondroitin.

In other preferred embodiments, the purified nutritional supplement or pharmaceutical is a pharmaceutical. These embodiments are not limited to any particular pharmaceutical. However, the skilled artisan would understand that the pharmaceutical cannot be very heat labile, or it will become inactivated upon brewing or steeping. The same understanding is present when a nutritional supplement is being utilized.

As used herein, a pharmaceutical is a chemical compound intended for use in the diagnosis, cure, mitigation, treatment, or prevention of disease in man or other animals, where the disease is not due to the lack of a naturally occurring compound in the man or animal.

These compositions are not limited to any particular binder. The skilled artisan could identify numerous such binders that are eatable and capable of adhering any particular nutritional supplement or pharmaceutical to the leaves of the plant material. Examples include lemon concentrate, starches (modified or unmodified), sugars, maltodextrins, carrageenan, gums, cellulose, waxes, corn syrup, peppermint oil, gelatin, soluble fiber, honey, almond extract, vanilla extract, chocolate bourbon, orange oil, aniseed oil, spearmint flavor, and strawberry flavor. In some preferred embodiments, the binder is lemon concentrate. In some more preferred embodiments, the plant material is green tea, the nutritional supplement or pharmaceutical is glucosamine, and the composition further comprises chondroitin.

The above compositions can be prepared as a loose tea, or bagged in a tea bag, which can be prepared by any known method, using any known materials. Preferably, the tea bag has green tea, glucosamine, and chondroitin.

The present invention is also directed to potable beverages resulting from steeping or brewing any of the above compositions. As used herein, steeping is soaking in water at a temperature below the boiling point. As used herein, brewing is soaking in water at or above the boiling point. It is understood that the water used for steeping or brewing should not be so hot as to inactivate the nutritional supplement or pharmaceutical.

In additional embodiments, the invention is directed to delivery systems for a nutritional supplement or pharmaceutical. In these embodiments, the delivery system comprises any of the above compositions in a tea bag, where the nutritional supplement or pharmaceutical is in a quantity suitable for unit dose administration. These delivery systems are useful for administering the nutritional supplement or pharmaceutical as a beverage. It is expected that these systems can be particularly useful for providing the nutritional supplement or pharmaceutical to a person or other animal (e.g., a dog, cat, bird) that has difficulty taking pills. In some preferred embodiments of these delivery systems, the plant material is green tea, the nutritional supplement or pharmaceutical is glucosamine, the binder is lemon concentrate, and the composition further comprises chondroitin.

In additional embodiments, the invention is directed to methods of ingesting a nutritional supplement or pharmaceutical. The methods comprise drinking the beverage resulting from steeping or brewing any of the above compositions.

Preferred embodiments of the invention are described in the following Example. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the Example.

### EXAMPLE

A green tea mixture was prepared by combining 272.0 pounds of decaffeinated green tea and 68.0 pounds of Soumee green tea in a blender. Lemon flavoring was then added to the blender as follows: 7.1 pounds of liquid lemon flavor #7140; 3.5 pounds of lemon #861077TD0990 WONF (Firmenich); and 3.5 pounds of lemon flakes #75F080246. After through blending, 159.4 pounds of a glucosamine chondroitin mixture was added. After blending, the mixture was air-dried for about two days. The mixture was then bagged into standard-sized tea bags (about three grams per bag). Each bag is calculated to have at least 0.5 g glucosamine and 0.4 g chondroitin.

The extract from one boiled tea bag was analyzed for glucosamine and chondroitin content by a commercial laboratory. Glucosamine content was determined to be 527.4 mg, as analyzed by the ALP-556-UV method. Chondroitin content was determined to be 596.6 mg, as determined by the ALP-579-LC method. This shows that there is no significant loss of glucosamine or chondroitin in the preparation, bagging, and brewing of the tea.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A composition suitable for steeping or brewing to form a potable beverage, the composition comprising plant material and at least one purified nutritional supplement or pharmaceutical adhering to the plant material with a binder.

2. The composition of claim 1, wherein the plant material is at least one of tea (Camillia sinensis) leaves, jasmine flowers, chamomile flowers, peppermint leaves, spearmint leaves, ginger, rose hips, orange peels, lemon grass, Roobois, Echinacea, or ginseng.

3. The composition of claim 1, wherein the plant material is tea (Camillia sinensis) leaves.

4. The composition of claim 3, wherein the tea (Camillia sinensis) is green tea, black tea, or white tea.

5. The composition of claim 3, wherein the tea (Camillia sinensis) is decaffeinated.

6. The composition of claim 1, wherein the nutritional supplement or pharmaceutical is a nutritional supplement.

7. The composition of claim 6, wherein the nutritional supplement is glucosamine, chondroitin, zinc, vitamin C, vitamin A, vitamin D, vitamin E, vitamin K, thiamine, riboflavin, niacin, vitamin B6, folic acid, vitamin B12, biotin, pantothenic acid, calcium, iron, magnesium, selenium, copper, manganese, chromium, potassium, methyl sulfonyl methane, P57, or lycopene.

8. The composition of claim 6, wherein the nutritional supplement is glucosamine or chondroitin.

9. The composition of claim 6, wherein the nutritional supplements are glucosamine and chondroitin.

10. The composition of claim 6, wherein the nutritional supplement is part of a multivitamin composition adhering to the plant material with a binder.

11. The composition of claim 1, wherein the nutritional supplement or pharmaceutical is a pharmaceutical.

12. The composition of claim 1, wherein the binder is lemon concentrate, a starch, corn syrup, a sugar, a maltodextrin, carrageenan, a gum, cellulose, a wax, peppermint oil, gelatin, soluble fiber, honey, almond extract, vanilla extract, chocolate bourbon, orange oil, aniseed oil, spearmint flavor, or strawberry flavor.

13. The composition of claim 1, wherein the binder is lemon concentrate.

14. The composition of claim 13, wherein the plant material is green tea, the nutritional supplement or pharmaceutical is glucosamine, and the composition further comprises chondroitin.

15. The composition of claim 1, in a tea bag.

16. The composition of claim 14, in a tea bag.

17. A potable beverage resulting from steeping or brewing the composition of claim 1.

18. A delivery system for a nutritional supplement or pharmaceutical, the delivery system comprising the composition and tea bag of claim 15, wherein the nutritional supplement or pharmaceutical is in a quantity suitable for unit dose administration.

19. The delivery system of claim 18, wherein the plant material is green tea, the nutritional supplement or pharmaceutical is glucosamine, the binder is lemon concentrate, and the composition further comprises chondroitin.

20. A method of ingesting a nutritional supplement or pharmaceutical, the method comprising drinking the beverage of claim 17.
